(19) United States
(12) Patent Application Publication
(10) Pub. No.: US 2011/0238041 A1
(43) Pub. Date: Sep. 29, 2011

(54) VARIABLE FLEXIBILITY CATHETER

(75) Inventors: Elaine Zhao Lim, Fremont, CA (US); Quang Quoc Tran, Redwood City, CA (US); Ting Tina Ye, San Jose, CA (US)

(73) Assignee: CHESTNUT MEDICAL TECHNOLOGIES, INC., Menlo Park, CA (US)

(21) Appl. No.: 12/731,110

(22) Filed: Mar. 24, 2010

(51) Int. Cl. A61M 25/00

(52) U.S. Cl. 604/527; 604/525

(57) ABSTRACT

A multi-layered catheter is provided, having a proximal end, a distal end, and a lumen extending therethrough. The catheter can have various sections of different flexibility extending distally along the length of the catheter. The catheter can have four layers along a proximal portion, and three layers along a distal portion. In some embodiments, the layers can comprise at least a stainless steel coil and a stainless steel braid. The catheter can exhibit both flexibility as well as kink resistance.

### FIELD OF THE INVENTIONS

The present application generally relates to catheters for use in the human body, and more specifically to multi-layered catheters having variable flexibility.

### BACKGROUND

Catheters, including microcatheters, are generally tubes inserted into the body through, for example a blood vessel, and have a variety of uses. Catheters generally have a proximal end, a distal end, and at least one lumen extending from the proximal to the distal end. Catheters can be used to deliver fluids, intra luminal devices such as stents, and/or other materials to a target location or locations inside the human body. Catheters suitable for a wide variety of applications are available commercially.

### SUMMARY

An aspect of at least one of the embodiments described herein includes the realization that small, flexible catheters often are difficult to maneuver within the tortuous pathways of the human anatomy, in particular the human neurovasculature. This is due to the fact that such catheters, and especially the intermediate and/or distal ends of such catheters, often bend, twist, and/or become entangled within the neurovasculature during medical procedures. This unwanted bending, twisting, and/or lack of control over the catheter can make it difficult to deliver intraluminal devices to specific locations in the human anatomy, such as for example an aneurysm in the neurovasculature.

Another aspect of at least one of the embodiments disclosed herein includes the realization that while relatively stiff and/or large catheters can overcome some of the problems associated with the bending and twisting described above, such catheters can be difficult to use, since they are often not flexible enough to be maneuvered through small, winding pathways inside the body.

It would thus be desirable to have a catheter which is small and flexible enough to be maneuvered through the narrow and winding pathways in the body, but also strong enough, stiff enough, and durable enough to resist unwanted bending or twisting, and to facilitate accurate delivery of fluids or intra luminal devices to specific target locations in body.

Therefore, in accordance with at least one embodiment, a variable flexibility catheter can comprise an elongate tubular body having a proximal end, a distal end, and an inner lumen extending therethrough. The elongate tubular body can comprise a proximal portion comprising a proximal portion outer jacket layer having a first stiffness, a braided stainless steel layer extending within the proximal portion outer jacket layer, a stainless steel coil layer extending within the braided material layer, and a low friction polymer PTFE layer extending within the stainless steel coil layer. The elongate tubular body can comprise an intermediate portion distal to the proximal portion comprising an intermediate portion outer jacket layer having a second stiffness, a portion of the braided stainless steel layer extending within the intermediate portion outer jacket layer, a portion of the stainless steel coil layer extending within the braided stainless steel layer, and a portion of the low friction polymer PTFE layer extending within the stainless steel coil layer. The elongate tubular body can comprise a taper portion distal to the intermediate portion comprising a tapered outer jacket layer having a third stiffness, a portion of the stainless steel coil layer extending within the tapered outer jacket layer, and a taper portion low friction polymer PTFE layer extending within the stainless steel coil layer. The elongate tubular body can comprise a distal portion distal to the taper portion comprising a distal portion outer jacket layer having a fourth stiffness, a portion of the stainless steel coil layer extending within the distal portion outer jacket layer, and a portion of the low friction polymer PTFE layer extending within the stainless steel coil layer. The second stiffness can be less than the first stiffness, the third stiffness can be less than the second stiffness, and the fourth stiffness can be less than the third stiffness. The stainless steel coil layer extending within the distal portion can have a coil pitch of approximately 0.007″ or more along at least one portion of the coil layer.

In accordance with another embodiment, a variable flexibility catheter can comprise an elongate tubular body having a proximal end, a distal end, and an inner lumen extending therethrough. The elongate tubular body can comprise a proximal portion comprising a proximal portion outer jacket layer having a first stiffness, a braided material layer extending within the proximal portion outer jacket layer, a coil layer extending within the braided material layer, and a low friction polymer material layer extending within the coil layer. The elongate tubular body can comprise an intermediate portion distal to the proximal portion comprising an intermediate portion outer jacket layer having a second stiffness, a portion of the braided material layer extending within the intermediate portion outer jacket layer, a portion of the coil layer extending within the braided material layer, and a portion of the low friction polymer material layer extending within the coil layer. The elongate tubular body can comprise a taper portion distal to the intermediate portion comprising a tapered outer jacket layer having a third stiffness, a portion of the coil layer extending within the tapered outer jacket layer, and a taper portion low friction polymer material layer extending within the coil layer. The elongate tubular body can comprise a distal portion distal to the taper portion comprising a distal portion outer jacket layer having a fourth stiffness, a portion of the coil layer extending within the distal portion outer jacket layer, and a portion of the low friction polymer material layer extending within the coil layer. The second stiffness can be less than the first stiffness, the third stiffness can be less than the second stiffness, and the fourth stiffness can be less than the third stiffness.

### BRIEF DESCRIPTION OF THE DRAWINGS

These and other features and advantages of the present embodiments will become more apparent upon reading the following detailed description and with reference to the accompanying drawings of the embodiments, in which:

FIG. 1 is a perspective view of an embodiment of a variable flexibility catheter;

FIG. 2 is a schematic illustration of the embodiment of FIG. 1, showing various separate sections along the length of the catheter;

FIG. 3 is a cross-sectional illustration of the catheter of FIG. 2;

FIG. 4 is an enlarged view of a section of the catheter of FIG. 3;

FIG. 5 is a cross-sectional view of the catheter section of FIG. 4;

FIG. 6 is a schematic illustration of the embodiment shown in FIG. 3, further illustrating various sections and lengths of the catheter embodiment;

FIG. 7 is a top, back, left side perspective illustration of an embodiment of a four-winged catheter hub for use with a variable flexibility catheter; and

FIG. 8 is a back side elevational view of the four-winged catheter hub of FIG. 7.

FIG. 9 is a schematic illustration of an embodiment of a variable flexibility catheter, showing delivery of an occluding device delivery system to an aneurysm in the neurovasculature.

FIG. 10 is an enlarged view of an embodiment of a variable flexibility catheter delivering an occluding device delivery system near an aneurysm.

FIG. 11 is a schematic illustration of an embodiment of a variable flexibility catheter, showing delivery of a clot retrieval device to a clot in the neurovasculature.

### DETAILED DESCRIPTION

### Variable Flexibility Catheter

An improved catheter 10 is disclosed herein. The embodiments disclosed herein are described in the context of a variable flexibility catheter for insertion into the human vasculature because the embodiments disclosed herein have particular utility in this context. However, the embodiments and inventions herein can also be applied to types of catheters (or catheters in general) configured for other type of environments.

The microcatheter 10 described herein is also described in the context of a catheter having a body comprised of four sections of varying flexibility extending distally along the catheter, the proximal end of the catheter comprising four layers, and the distal end of the catheter comprising three layers, with a central lumen extending the length of the catheter. However, the embodiments and inventions of the catheters described herein can include various other combinations and numbers of sections, layers, and/or lumens. Thus, it is to be understood that the embodiments and inventions described herein are not limited to any one combination.

In particular, at least one of the embodiments of the catheter 10 described herein is described as having a proximal portion, a strain relief jacket surrounding the proximal portion, a catheter hub releasably attached to the proximal portion, at least one intermediate portion located distal of the proximal portion, at least one taper portion located distal of the proximal portion, and a distal portion.

### Proximal Portion

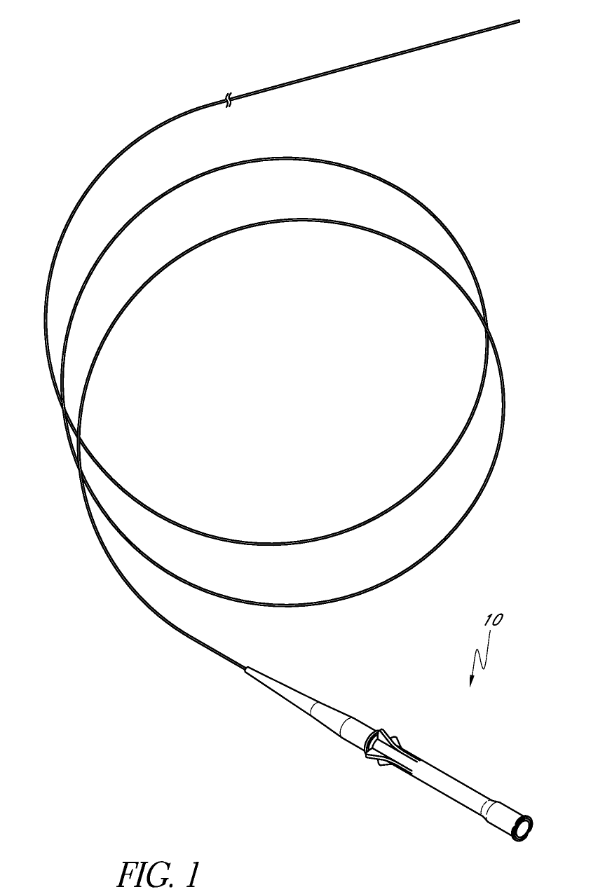

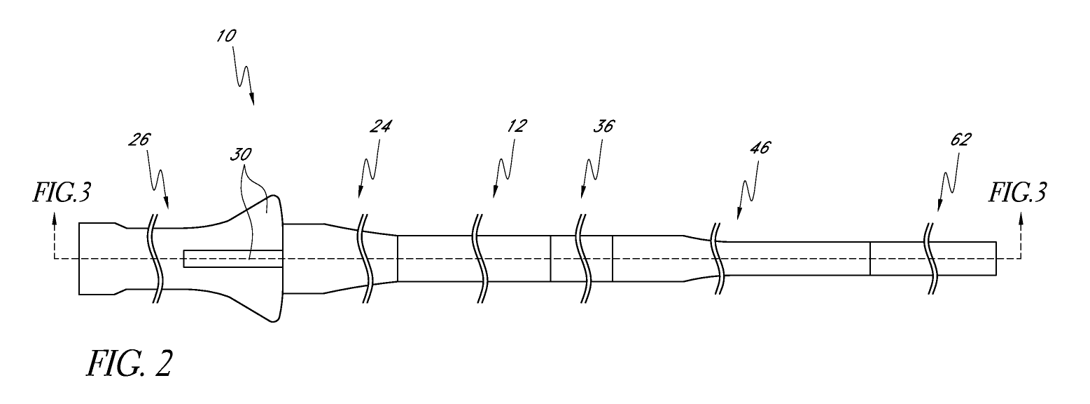

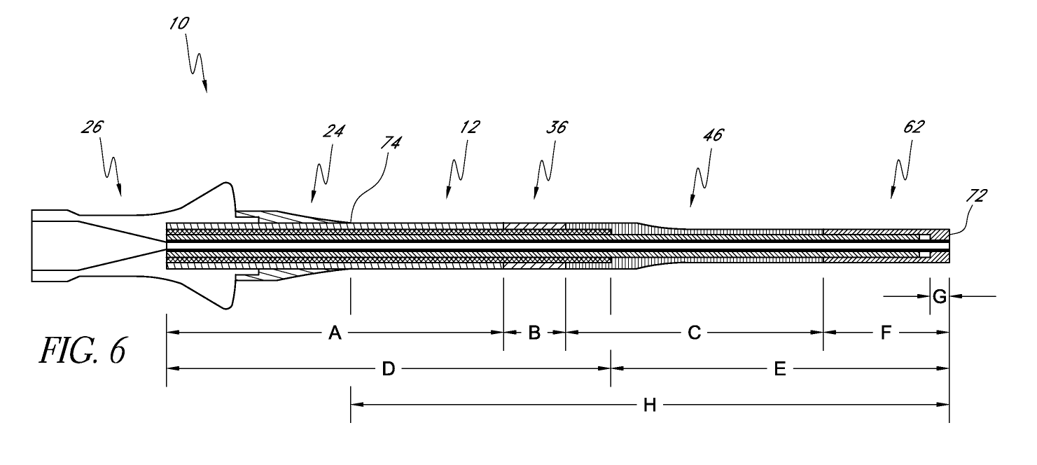

With reference to FIGS. 1-6, and as described above, the catheter 10 can comprise a proximal portion 12. The proximal portion 12 can vary in length. FIG. 6 illustrates a length “A” for the proximal portion 12. In a preferred arrangement, the length “A” of the proximal portion 12 can range between approximately 58 cm and 113 cm, though other ranges are also possible.

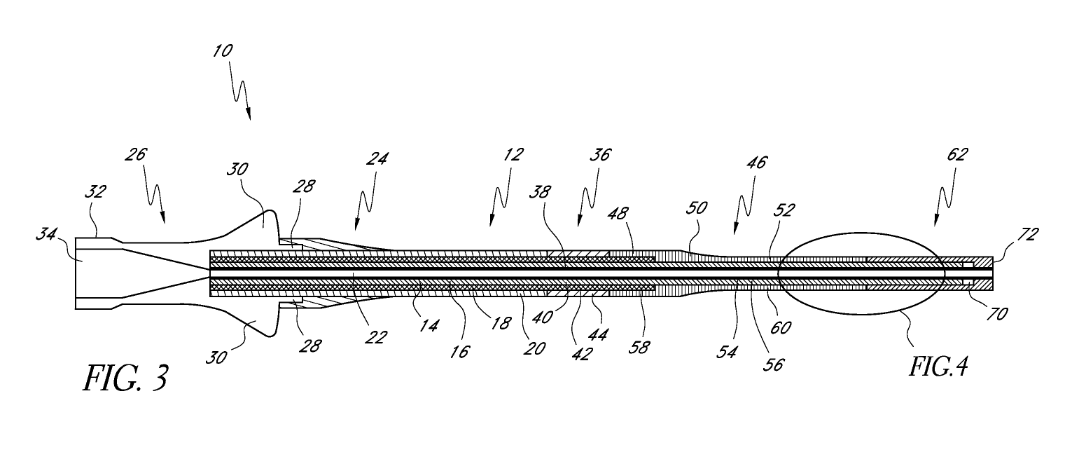

With reference to FIGS. 3 and 5, the proximal portion 12, along with other portions of the catheter 10 described herein, can have a generally circular-shaped cross section such as that shown in FIG. 5. However, other cross-sectional shapes are also possible. The proximal portion 12 can have an outer diameter. In some embodiments the outer diameter can remain constant along the length of the proximal portion 12. In some embodiments, the outside diameter of the proximal portion 12 can range between 0.040″ and 0.044″, though other ranges are also possible.

With continued reference to FIG. 3, the proximal portion 12 can comprise at least one layer. In a preferred arrangement, the proximal portion 12 can comprise four layers. For example, the proximal portion 12 can comprise a first proximal portion layer 14, a second proximal portion layer 16, a third proximal portion layer 18, and a fourth proximal portion layer 20 as shown in FIG. 3. The first, second, third, and fourth proximal portion layers 14, 16, 18, and 20 can surround an internal lumen 22.

In some embodiments, the first proximal portion layer 14 can comprise a low friction polymer material layer extending for at least a portion of the length of proximal portion 12. In a preferred arrangement, the low frictional polymer material can comprise an extruded, etched, PTFE tubing that also extends distally beyond the proximal portion 12. The PTFE tubing can form a thin inner liner within the catheter 10. The PTFE liner can inhibit friction within the catheter, such as for example during delivery of intra luminal devices through the catheter's lumen 22 (See, for example, U.S. Patent Publication No. 2006/0271149, U.S. Patent Publication No. 2006/0271153, U.S. Patent Publication No. 2009/0318947, U.S. Pat. No. 6,679,893, and U.S. Patent Publication No. 2008/0269774, the entirety of each of which is hereby incorporated by reference, for non-limiting examples of intra luminal devices that can be used with the catheter 10 described herein). This reduction in friction can help to reduce the force required to deliver an intra luminal device through the catheter 10 (e.g., to push the intraluminal device through the catheter 10 towards a target location in the human body), or to more easily slide the catheter 10 over a guidewire extending through the lumen 22.

Additionally, the thickness of the first proximal portion layer 14 can be optimized so that the first proximal portion layer 14 is durable enough to withstand radial forces exerted by intra luminal devices as they are delivered through the catheter, yet still flexible enough to allow the catheter 10 to negotiate through challenging anatomies, such as for example the narrow and winding neurovasculature of a patient's brain. In some embodiments, the thickness of the first proximal portion layer 14 can range between approximately 0.0005″ and 0.0012″, though other ranges are also possible. In a preferred arrangement, the thickness of the first proximal portion layer 14 can be approximately 0.001″.

With continued reference to FIG. 3, in some embodiments the second proximal portion layer 16 can comprise a coil layer extending for at least a portion of the length of the proximal portion 12, and preferably distally beyond the proximal portion 12. The coil layer can provide strength and/or kink resistance along the length of proximal portion 12. In a preferred arrangement, coil layer can surround the first proximal portion layer 14 and can comprise a stainless steel coil layer comprised of a single wound stainless steel coil. Other types of metals or materials are also possible for the coil layer, as are other numbers of coils. The coil or coils forming the coil layer can have a generally circular cross-section, though other cross-sectional shapes are also possible. In some embodiments, the cross-sectional diameter of the coil can range between approximately 0.0014″ and 0.0016″, though other ranges are also possible. In a preferred arrangement, the cross-sectional diameter of the coil can be approximately 0.0015″.

In some embodiments, the coil can have a varying pitch. For example, the pitch of the coil can decrease moving distally down the proximal portion 12. In a preferred arrangement, the coil can have a pitch of between approximately 0.016″ and 0.018″ at the most proximal end of proximal portion 12. In some embodiments the pitch can remain between approximately 0.016″ and 0.018″ moving distally along the proximal portion 12 for a predetermined length of the proximal portion 12, at which point the pitch can then decrease to somewhere between 0.012″ and 0.014″, and then decrease further to somewhere between 0.010″ and 0.012″ at a more distal location along the proximal portion 12. Other pitch lengths and/or ranges are also possible. In some embodiments, the pitch of the coil can remain constant throughout the proximal portion 12. Furthermore, in some embodiments, rather than decreasing in pitch, the coil forming the second proximal portion layer 16 can increase in length moving distally down the proximal portion 12.

With continued reference to FIG. 3, in some embodiments the third proximal portion layer 18 can comprise a braid layer extending for at least a portion of the length of the proximal portion 12, and preferably distally beyond the proximal portion 12. In a preferred arrangement, the braid layer can surround the second proximal portion layer 16 and can comprise a stainless steel braid layer. Other types of metals or materials are also possible for the braid layer. In some embodiments, the braid layer can have a uniform density extending along its entire length. In a preferred arrangement, the each of the braid strands can have a thickness of approximately 0.007″ and a width of approximately 0.0025″, though other values and ranges are also possible. The combination of the braid layer with the coil layer can add additional strength and rigidity to the proximal portion 12 of catheter 10. For example, the strength added of the braid layer can facilitate greater pushability of the proximal portion 12, pushability relating generally to the ease with which one can push the proximal portion 12 through the human anatomy without unwanted flexion and/or movement of the proximal portion 12.

With continued reference to FIG. 3, in some embodiments the fourth proximal portion layer 20 can comprise a proximal portion outer jacket layer extending for at least a portion of the length of the proximal portion 12, and in the illustrated embodiment has a proximal and a distal end that correspond to the proximal and distal ends of the proximal portion 12. In a preferred arrangement, the proximal portion outer jacket layer can surround the third proximal portion layer 18, and can comprise a polymer layer, such as for example a plastic resin like Pebax. Other types of materials are also possible for the proximal portion outer jacket layer, including but not limited to polyurethane. In some embodiments, the proximal portion outer jacket layer can have a smooth outer diameter profile. The proximal portion outer jacket layer can comprise a hydrophilic coating to provide a smooth outer surface, thereby reducing friction and facilitating ease of catheter delivery into the human anatomy. The hydrophilic coating can be any commonly used hydrophilic coatings in the industry. The proximal portion outer jacket layer can further have a stiffness that helps give the proximal portion 12 more rigidity and strength than other portions of the catheter 10. In some embodiments, the proximal portion outer jacket layer can comprise Pebax 7233-B20, which when combined with the additional layers of proximal portion 12 can give the proximal portion 12 a stiffness that measures approximately 1.06 gm, though other measurements and ranges are also possible.

With continued reference to FIG. 3, the lumen 22 can extend the entire length of catheter 10. In some embodiments, the lumen 22 can have a constant diameter. In a preferred arrangement, the lumen 22 can have a diameter ranging between approximately 0.026″ and 0.028,″ though other ranges are also possible.

### Strain Relief Jacket

With reference to FIGS. 1-3, the catheter 10 can comprise a strain relief jacket 24. The strain relief jacket 24 can comprise, for example, a tubular hollow structure attached to or forming part of the proximal portion 12. For example, the strain relief jacket 24 can be integrally formed on an outside portion of the proximal portion 12. The strain relief jacket 24 can act as a bridge between the hub and the proximal portion 12 of the catheter to protect the proximal portion 12 from kinking. The strain relief jacket 24 can add structural rigidity to one end of the catheter 10. In some embodiments, the strain relief jacket 24 can have a generally tapered outer diameter, decreasing in diameter distally along the catheter. The strain relief jacket can comprise a polymer, including but not limited to Santoprene 45A.

### Catheter Hub

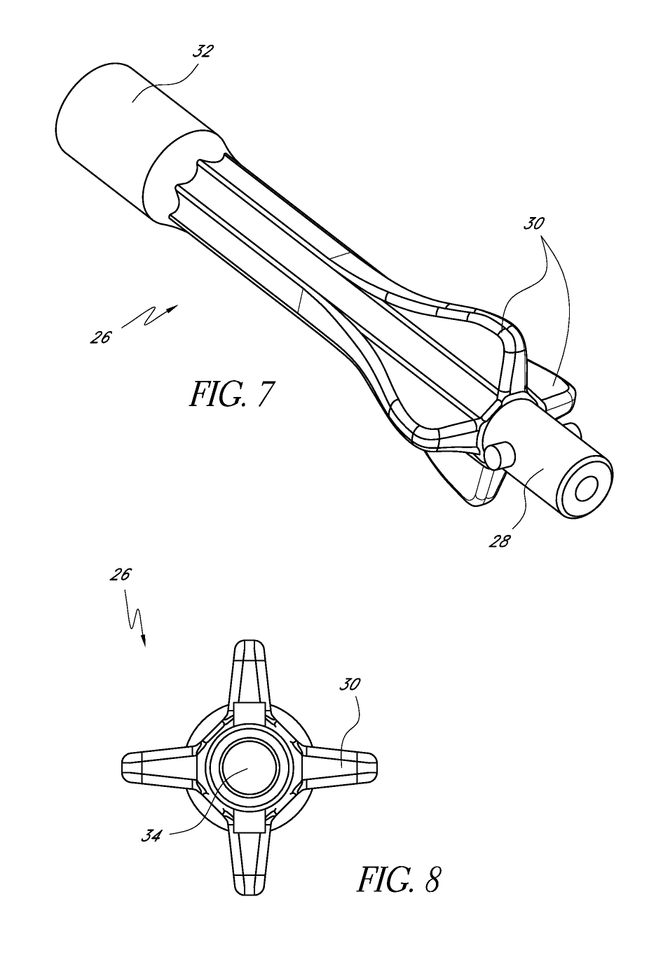

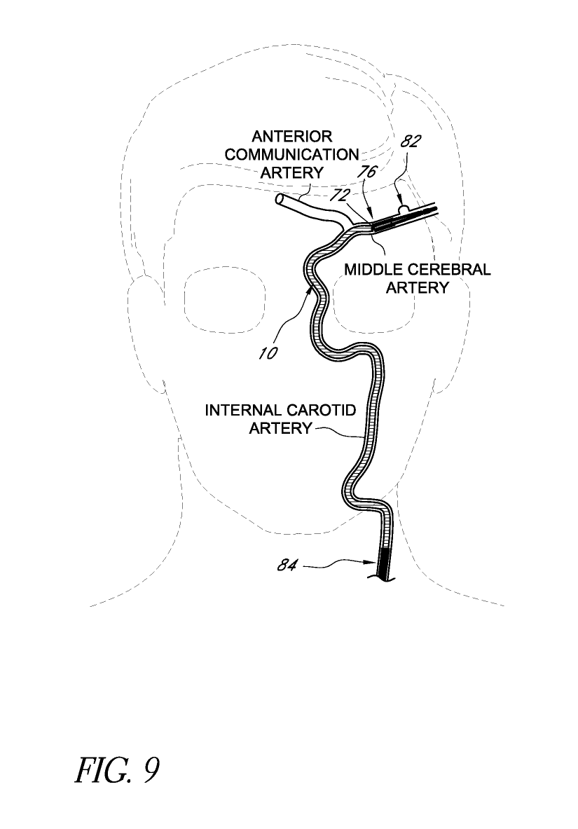

With reference to FIGS. 1-3, 7, and 8, the catheter 10 can comprise a catheter hub 26. The catheter hub 26 can be attached to another portion of the catheter 10. For example, the catheter hub 26 can comprise a distal end 28 that is attached to (e.g., via an interference fit, adhesion, bonding, any other type of attachment) the strain relief jacket 24 and/or the proximal portion 12 of the catheter 10. As illustrated in FIG. 3, in some embodiments the proximal portion 12 can extend at least partially within the hub 26. In some embodiments, the catheter hub 26 can be attached to the rest of the catheter 10.

The catheter hub 26 can comprise at least one gripping structure or structures for easy manipulation and handling (e.g., twisting or turning of the hub 26 and/or catheter 10). For example, the catheter hub 26 can comprise at least one hub wing 30. In a preferred arrangement, the catheter hub 26 can comprise four hub wings 30. The four hub wings 30 can be spaced equidistantly from one another circumferentially around the hub 26. The hub wings 30 can be gripped by hand, for example, to turn or move the hub 26 and/or catheter 10.

The catheter hub 26 can further comprise a proximal end 32 having an open cavity 34 extending therethrough, preferably tapered distally. The open cavity 34 can be used, for example, to direct fluid, material, or another device or devices into or through the catheter 10.

In a preferred arrangement, the combination catheter hub 26 can have an overall length of approximately 1.9″ and the strain relief jacket 24 can have an overall length of approximately 1.36″, though other lengths and ranges are also possible.

### Intermediate Portion(s)

With reference to FIGS. 1-6, the catheter 10 can comprise at least one intermediate portion 36. The intermediate portion 36 can vary in length. FIG. 6 illustrates a length “B” for the intermediate portion 36. In a preferred arrangement, the length “B” of the intermediate portion 36 can range between approximately 8.5 cm and 11.5 cm, though other ranges are also possible.

With reference to FIGS. 3 and 5, the intermediate portion 36, along with the other portions of the catheter 10 described herein, can have a generally circular-shaped cross section such as that shown in FIG. 5. However, other cross-sectional shapes are also possible. The intermediate portion 36 can have an outer diameter. In some embodiments the outer diameter can remain constant along the length of the intermediate portion 36 and be the same as the outer diameter of the proximal portion 12. In some embodiments, the outside diameter of the intermediate portion 36 can range between 0.040″ and 0.044″, though other ranges are also possible. In a preferred arrangement, the outer diameter of the intermediate portion 36 can be approximately 0.042″.

With continued reference to FIG. 3, the intermediate portion 36 can comprise at least one layer. In a preferred arrangement, the intermediate portion 36 can comprise four layers. For example, the intermediate portion 36 can comprise a first intermediate portion layer 38, a second intermediate portion layer 40, a third intermediate portion layer 42, and a fourth intermediate portion layer 44 as shown in FIG. 3. The first, second, third, and fourth intermediate portion layers 38, 40, 42, and 44 can surround the internal lumen 22.

In a preferred arrangement, the first intermediate portion layer 38 can comprise the same layer of extruded, etched, PTFE tubing as in the first proximal portion layer 14. Thus, the first proximal portion layer 14 and first intermediate portion layer 38 can together comprise a single inner liner of PTFE material extending along both the proximal portion 12 and intermediate portion 36. However, in other embodiments the first intermediate portion layer 38 can be comprised of a different material or structure than that of first proximal portion layer 14.

Additionally, the thickness of the first intermediate portion layer 38 can be optimized so that the first intermediate portion layer 38 is durable enough to withstand radial forces exerted by intra luminal devices as they are delivered through the catheter, yet still flexible enough to allow the catheter 10 to negotiate through challenging anatomies, such as for example the narrow and winding neurovasculature of a patient's brain. In some embodiments, the thickness of the first intermediate portion layer 38 can range between approximately 0.0005″ and 0.0012″, though other ranges are also possible. In a preferred arrangement, the thickness of the first intermediate portion layer 38 can be approximately 0.001″.

In a preferred arrangement, the second intermediate portion layer 40 can comprise the same coil layer as in the second proximal portion layer 16. Thus, the second proximal portion layer 16 and second intermediate portion layer 40 can together comprise a single stainless steel coil extending along both the proximal portion 12 and intermediate portion 36. However, in other embodiments the second intermediate portion layer 40 can be comprised of a different material or structure than that of second proximal portion layer 16.

In some embodiments, the coil in the second intermediate portion layer 40 can have a varying pitch. For example, the pitch of the coil can decrease moving distally down the intermediate portion 36. In other embodiments the coil can have a constant pitch, or can increase moving distally down the intermediate portion 36. In a preferred arrangement, the coil can have a pitch of between approximately 0.008″ and 0.018″ within the intermediate portion 36, though other ranges are also possible.

In a preferred arrangement, the third intermediate portion layer 42 can comprise the same braid layer as in the third proximal portion layer 18. Thus, the third proximal portion layer 18 and third intermediate portion layer 42 can together comprise a single stainless steel braid layer extending along both the proximal portion 12 and intermediate portion 36. However, in other embodiments the third intermediate portion layer 42 can be comprised of a different material or structure than that of third proximal portion layer 18.

With continued reference to FIG. 3, the fourth intermediate portion layer 44 can comprise an outer jacket layer extending for at least a portion of the length of the intermediate portion 36. In a preferred arrangement, the outer jacket layer can surround the third intermediate portion layer 42, and can comprise a material that is less stiff than the material forming the proximal portion outer jacket layer described above. In some embodiments, the intermediate portion outer jacket layer can comprise Pebax, though other types of materials are also possible. The intermediate portion outer jacket layer can have a smooth outer diameter profile, and can comprise a hydrophilic coating to provide a smooth outer surface. The intermediate portion outer jacket layer can further have a specific stiffness. In some embodiments, the intermediate portion outer jacket layer can comprise Pebax 5533-B20, which has a stiffness less than that of Pebax 7233-B20. This reduction in stiffness from the proximal portion 12 to the intermediate portion 36 can give the catheter 10 more flexibility in the intermediate portion. However, due to the internal coil and braid layers, the intermediate portion 36 can still advantageously retain a level of stiffness and rigidity that enables a user to easily guide and push the catheter 10 through the human anatomy.

### Taper Portion(s)

With reference to FIGS. 1-6, the catheter 10 can comprise at least one taper portion 46. The taper portion 46 can vary in length. FIG. 6 illustrates a length “C” for the taper portion 46. In a preferred arrangement, the length “C” of the taper portion 46 can range between approximately 6 cm and 33 cm, though other ranges are also possible.

With reference to FIGS. 3 and 5, the taper portion 46, along with the other portions of the catheter 10 described herein, can have a generally circular-shaped cross section such as that shown in FIG. 5. However, other cross-sectional shapes are also possible. The taper portion 46 can further comprise an outer diameter. In a preferred arrangement, the taper portion 46 can comprise a first segment 48, a second segment 50 located distal of the first segment 48, and a third segment 52 located distal of the second segment 50. In some embodiments the first segment 48 can have an outer diameter similar or identical to the outer diameter of the intermediate portion 36, the second segment 50 can have a tapering diameter that decreases in size between the first and third segments 48, 52, and the third segment 52 can have a generally constant diameter less than that of the first segment 48.

In a preferred arrangement, the outer diameter of the third segment 52 can range between approximately 0.034″ and 0.038″, though other ranges are also possible. Additionally, in a preferred arrangement, the length of the first segment 48 can range from 2.5-3 cm, the length of the second segment 50 can range from 1.5-3.5 cm, and the length of the third segment 52 can range from 0.5-27.5 cm, though other ranges are also possible.

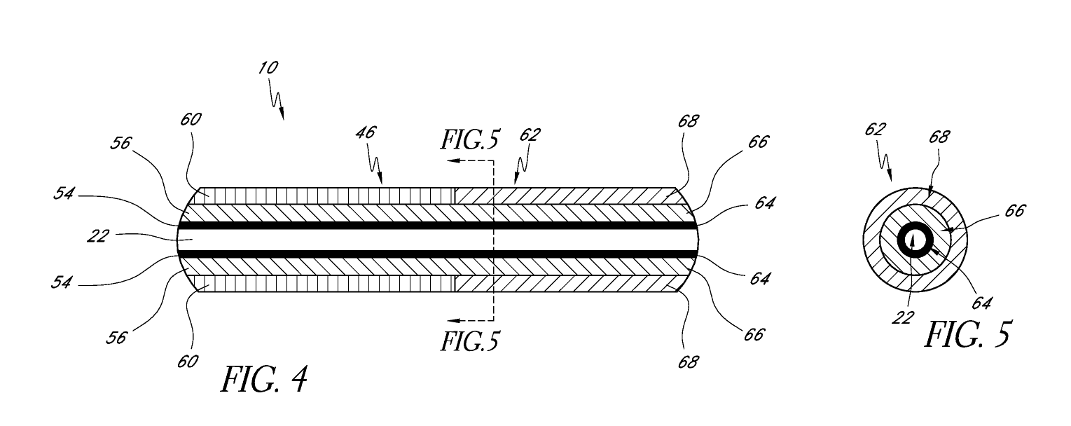

With continued reference to FIGS. 3 and 4, the taper portion 46 can comprise at least one layer. In a preferred arrangement, the taper portion 46 can comprise four layers in one segment of the taper portion 46, and three layers in a more distal segment of the taper portion 46. For example, the taper portion 46 can comprise four layers in the first segment 48, and three layers in the second and/or third segments 50, 52.

With continued reference to FIG. 3, the taper portion 46 can comprise a first taper portion layer 54, a second taper portion layer 56, a third taper portion layer 58, and a fourth taper portion layer 60 as shown in FIGS. 3 and 4. The first, second, third, and fourth taper portion layers 54, 56, 58, and 60 can surround the internal lumen 22.

In a preferred arrangement, the first taper portion layer 54 can comprise the same layer of extruded, etched, PTFE tubing as in the first proximal portion layer 14 and first intermediate portion layer 38. Thus, the first proximal portion layer 14, first intermediate portion layer 38, and first taper portion layer 54 can together comprise a single inner liner of PTFE material extending along the proximal portion 12, intermediate portion 36, and taper portion 46. However, in other embodiments the first taper portion layer 54 can be comprised of a different material or structure than that of first proximal portion layer 14 or first intermediate portion layer 38.

Additionally, the thickness of the first taper portion layer 54 can be optimized so that the first taper portion layer 54 is durable enough to withstand radial forces exerted by intra luminal devices as they are delivered through the catheter, yet still flexible enough to allow the catheter 10 to negotiate through challenging anatomies, such as for example the narrow and winding neurovasculature of a patient's brain. In some embodiments, the thickness of the first taper portion layer 54 can range between approximately 0.0005″ and 0.0012″, though other ranges are also possible. In a preferred arrangement, the thickness of the first taper portion layer 54 can be approximately 0.001″.

In a preferred arrangement, the second taper portion layer 56 can comprise the same coil layer as in the second proximal portion layer 16 and second intermediate portion layer 40. Thus, the second proximal portion layer 16, second intermediate portion layer 40, and second taper portion layer 56 can together comprise a single stainless steel coil extending along the proximal portion 12, intermediate portion 36, and taper portion 46. However, in other embodiments the second taper portion layer 56 can be comprised of a different material or structure than that of second proximal portion layer 16 or second intermediate portion layer 40.

In some embodiments, the coil in the second taper portion layer 56 can have a varying pitch. For example, the pitch of the coil can decrease moving distally down the taper portion 46. In other embodiments the coil can have a constant pitch, or can increase moving distally down the taper portion 46. In a preferred arrangement, the coil can have a pitch of between approximately 0.007″ and 0.012″ within the intermediate portion 36, though other ranges are also possible.

In a preferred arrangement, the third taper portion layer 58 can comprise the same braid layer as in the third proximal portion layer 18 and third intermediate portion layer 42. Thus, the third proximal portion layer 18, third intermediate portion layer 42, and third taper portion layer 58 can together comprise a single stainless steel braid layer extending along the proximal portion 12, intermediate portion 36, and at least a portion of the taper portion 46. However, in other embodiments the third intermediate portion layer 42 can be comprised of a different material or structure than that of third proximal portion layer 18.

As illustrated in FIGS. 3 and 6, in a preferred arrangement, the third taper portion layer 58 can extend along at least a portion of the first segment 48, but not along segments 50 and 52. Thus, a braid layer in catheter 10 can end proximate of a point where the outside diameter of taper portion 46 begins to decrease. For example, with reference to FIG. 6, the braid layer can extend a distance “D” along the catheter 10. In a preferred arrangement, the distance “D” can range from approximately 65 cm to 110 cm, though other ranges are also possible. The distance “E” illustrated in FIG. 6 can be the length of the catheter 10 that does not comprise a braid layer. The distance “E” can range from approximately 13-52 cm, though other ranges are also possible.

With continued reference to FIG. 3, the fourth taper portion layer 60 can comprise an outer jacket layer extending for at least a portion of the length of the fourth taper portion layer 60. In a preferred arrangement, the outer jacket layer can surround the third taper portion layer 58, and can comprise a material that is less stiff than the material forming the proximal portion outer jacket layer and intermediate portion outer jacket layer described above. In some embodiments, the taper portion outer jacket layer can comprise Pebax, though other types of materials are also possible. The taper portion outer jacket layer can have a smooth outer diameter profile, and can comprise a hydrophilic coating to provide a smooth outer surface. The taper portion outer jacket layer can further have a specific stiffness. In some embodiments, the taper portion outer jacket layer can comprise Pebax 4033-B20, which has a stiffness less than that of Pebax 5533-B20 and Pebax 7233-B20. This reduction in stiffness from the proximal portion 12, to the intermediate portion 36, to the taper portion 46, can give the catheter 10 more flexibility in the taper portion 46 than in the proximal or intermediate portions 12 and 36. Furthermore, the reduction from four layers to three layers in the taper portion 46 can provide the catheter 10 with more flexibility in the taper portion 46 than in any of the more proximal portions, yet still provide the catheter 10 with enough stiffness and rigidity to move through the vasculature and easily be pushed and manipulated through difficult (e.g., winding) passageways in the human anatomy.

### Distal Portion

With reference to FIGS. 1-6, the catheter 10 can comprise a distal portion 62. The distal portion 62 can vary in length. FIG. 6 illustrates a length “F” for the distal portion 62. In a preferred arrangement, the length “F” of the distal portion 62 can range between approximately 4 cm and 21 cm, though other ranges are also possible.

With reference to FIGS. 3 and 5, the distal portion 62, along with the other portions of the catheter 10 described herein, can have a generally circular-shaped cross section such as that shown in FIG. 5. However, other cross-sectional shapes are also possible. The distal portion 62 can further comprise an outer diameter. In a preferred arrangement, the outer diameter of the distal portion 62 can range between approximately 0.034″ and 0.038″, though other ranges are also possible.

With continued reference to FIGS. 3-5, the distal portion 62 can comprise at least one layer. In a preferred arrangement, the distal portion 62 can comprise three layers. The distal portion 62 can comprise a first distal portion layer 64, a second distal portion layer 66, and a third distal portion layer 68 as shown in FIGS. 4 and 5. The first, second, and third distal portion layers 64, 66, and 68 can surround the internal lumen 22.

In a preferred arrangement, the first distal portion layer 64 can comprise the same layer of extruded, etched, PTFE tubing as in the first proximal portion layer 14, the first intermediate portion layer 38, and the first taper portion layer 54. Thus, the first proximal portion layer 14, first intermediate portion layer 38, first taper portion layer 54, and first distal portion layer 64 can together comprise a single inner liner of PTFE material extending along the proximal portion 12, intermediate portion 36, taper portion 46, and distal portion 62. However, in other embodiments the first distal portion layer 64 can be comprised of a different material or structure than that of first proximal portion layer 14, first intermediate portion layer 38, or first taper portion layer 54.

Additionally, the thickness of the first distal portion layer 64 can be optimized so that the first distal portion layer 64 is durable enough to withstand radial forces exerted by intra luminal devices as they are delivered through the catheter, yet still flexible enough to allow the catheter 10 to negotiate through challenging anatomies, such as for example the narrow and winding neurovasculature of a patient's brain. In some embodiments, the thickness of the first distal portion layer 64 can range between approximately 0.0005″ and 0.0012″, though other ranges are also possible. In a preferred arrangement, the thickness of the first distal portion layer 64 can be approximately 0.001″.

In a preferred arrangement, the second distal portion layer 66 can comprise the same coil layer as in the second proximal portion layer 16, second intermediate portion layer 40, and second taper portion layer 56. Thus, the second proximal portion layer 16, second intermediate portion layer 40, second taper portion layer 56, and second distal portion layer 66 can together comprise a single stainless steel coil extending along the proximal portion 12, intermediate portion 36, taper portion 46, and distal portion 62. However, in other embodiments the second distal portion layer 66 can be comprised of a different material or structure than that of second proximal portion layer 16, second intermediate portion layer 40, or second taper portion layer 56.

In some embodiments, the coil in the second distal portion layer 66 can have a varying pitch. For example, the pitch of the coil can decrease moving distally down the distal portion 62. In other embodiments the coil can have a constant pitch, or can increase moving distally down the distal portion 62. In a preferred arrangement, the coil can have a pitch of between approximately 0.007″ and 0.009″ within the distal portion 62, although other pitches and ranges of pitches are also possible.

Furthermore, in some embodiments, the distal portion 62 can comprise at least one marker band 70, and a distal tip 72 (e.g., an atraumatic tip having smoothed edges to prevent vessel damage within the body). In a preferred arrangement, the distal tip 72 can comprise a polymer, more particularly a plastic resin such as Pebax 2533. With reference to FIG. 3, the second distal portion layer 66 can extend partially along the distal portion 62 before it ends at the marker band 70. FIG. 6 illustrates a length “G”, the distance between the marker band 70 and tip 72. The length “G” can range between approximately 0.5 mm and 1.0 m. Other lengths or ranges of lengths are also possible.

The marker band 70 can comprise, for example, a metal or metal alloy ring such as platinum, Nitinol and/or a gold ring which can be visualized via fluoroscopy. During use of the catheter 10, a surgeon or other medical personnel may find it helpful to know where the tip 72 of the catheter 10 is in relation to a desired target location (e.g., an aneurysm in the neurovasculature). If the surgeon or other medical personnel is aware of the tip's location, he or she can maneuver the catheter 10 so as to deploy an intra luminal device precisely at a given target location based on knowledge of the marker band's (and consequently the tip's) location.

With continued reference to FIG. 3, the third distal portion layer 68 can comprise an outer jacket layer extending for at least a portion of the length of the third distal portion layer 68. In a preferred arrangement, the outer jacket layer can surround the second distal portion layer 66, and can comprise a material that is less stiff than the material forming the proximal portion outer jacket layer, intermediate portion outer jacket layer, and taper portion layer described above. In some embodiments, the distal portion outer jacket layer can comprise Pebax, though other types of materials are also possible. The distal portion outer jacket layer can have a smooth outer diameter profile, and can comprise a hydrophilic coating to provide a smooth outer surface. The distal portion outer jacket layer can further have a specific stiffness. In some embodiments, the distal portion outer jacket layer can comprise Pebax 2533-B20, which has a stiffness less than that of Pebax 4033-B20, Pebax 5533-B20, and Pebax 7233-B20. In some embodiments, the distal portion outer jacket layer, when combined with the additional layers of distal portion 62, can give the distal portion 62 a stiffness that measures approximately 0.089 gm, though other measurements and ranges are also possible.

This reduction in stiffness from the proximal portion 12, to the intermediate portion 36, to the taper portion 46, to the distal portion 62 can give the catheter 10 more flexibility in the distal portion 62 than in the proximal, intermediate, or taper portions 12, 36, and 46. Furthermore, having three layers in the distal portion 62 can provide the catheter 10 with more flexibility in the distal portion 62 than in any of the more proximal portions, yet still provide the catheter 10 with enough stiffness and rigidity to move through the vasculature and easily be pushed and manipulated through difficult (e.g., winding) passageways in the human anatomy.

With continued reference to FIG. 6, the catheter 10 can further comprise a working length “H” that extends from a distal end 74 of the strain relief jacket 24 to the distal tip 66. In a preferred arrangement, the working length “H” can range, for example, from approximately 77 cm to 153 cm, though other ranges are also possible.

### Assembly

To construct the catheter as illustrated in FIGS. 3 and 4, the second proximal portion layer 16, second intermediate portion layer 40, second taper portion layer 56, and second distal portion layer 66 (which as described can be a single coil stainless layer) can be placed around the first proximal portion layer 14, first intermediate portion layer 38, first taper portion layer 54, and first distal portion layer 64 (which as described can be a single layer of low friction PTFE) using a winding machine. For example, in a preferred arrangement, the etched PTFE liner described above can be placed on a mandrel. While still on the mandrel, a stainless steel coil can be wound on top of the etched PTFE liner using a common coil winding machine. The coil winding machine can wind the coil at specified pitches along the proximal portion 12, intermediate portion 36, taper portion 46, and distal portion 62. In a preferred arrangement, the stainless steel coil pitch can be wound constant for a specified length of the catheter moving proximally along the catheter, at which point the winding then changes to a wider pitch, and then to an even wider pitch, etc. Thus, in a preferred arrangement, the pitch of the stainless steel coil can be lowered in increments moving down the catheter 10, and can have a pitch within the ranges described above in each of the proximal, intermediate, taper, and distal portions 12, 36, 46, and 62.

The third proximal portion layer 18, third intermediate portion layer 42, and third taper portion layer 58 (which as described can be a single braided stainless steel layer) can then be placed around the second proximal portion layer 16, second intermediate portion layer 40, and second taper portion layer 56. For example, in a preferred arrangement, the stainless steel braid described above can be created using a Steeger Braider. In a preferred arrangement each of the stainless steel strands braided together can have a thickness of approximately 0.0007″ and a width of approximately 0.0025″, though other values and ranges are also possible. While the catheter 10 is still on the mandrel, the stainless steel braid can be stretched proximally over the catheter 10, and cut to a specified length “D”.

The fourth proximal portion layer 20, fourth intermediate portion layer 44, fourth taper portion layer 58, and third distal portion layer 68, which in a preferred arrangement can each comprise Pebax, can then be added. Each of the fourth proximal portion layer 20, fourth intermediate portion layer 44, fourth taper portion layer 58, and third distal portion layer 68 can for example be extruded, and can be pulled onto (e.g. slid over) the rest of the catheter assembly, and then heat shrunk in place. Each of the fourth proximal portion layer 20, fourth intermediate portion layer 44, fourth taper portion layer 58, and third distal portion layer 68 can have a different stiffness as described above so that the catheter 10 is more flexible at a distal end than at a proximal end.

### Further Catheter Advantages

As described above, the embodiments of the catheter 10 can have a coil layer, and in particular a stainless steel coil layer, which extends substantially the entire length of the catheter 10. The coil layer can comprise a single wound stainless steel coil having a circular cross section. Furthermore, the coil can have varying pitch. In a preferred arrangement, the pitch of the stainless steel coil can decrease moving distally along the catheter 10. Thus, while the catheter 10 overall can increase in flexibility moving distally along the catheter (e.g., due to the outer jacket layers comprised of material which has a lower hardness in each portion moving distally along the catheter 10, and the number of layers and overall outer diameter of the catheter 10 decreasing moving distally along the catheter 10), the distal portion 62 and area surrounding the tip 72 can be flexible enough, and strong enough, to withstand kinking of the distal portion 62. Kinking, as described herein, refers generally to the outside diameter of the catheter 10 decreasing in size along at least one axis due to twisting or manipulation of the catheter 10. For example, the distal portion 62 of catheter 10 can have a generally circular cross-section, as shown in FIG. 3. If the distal portion 62 is bent, twisted, or wrapped about an object, the distal portion 62 can tend to kink, and the circular cross-section can take on more of an oval shape. Thus, along at least one axis, the outside diameter will decrease, making it more difficult to push intra luminal devices through the distal portion 62.

In some embodiments, it has been found that having a stainless steel coil of the type described above, with a pitch diameter of approximately 0.007″-0.009″ along the coil's most distal end, can facilitate a kink resistance of at least 75% based on a first kink resistance test. In some embodiments, the kink resistance can be at least 85% based on a first kink resistance test. In some embodiments, the kink resistance can be at least 95% based on a first kink resistance test. In some embodiments, the kink resistance can be at least 98% based on a first kink resistance test. The first kink resistance test can comprise, for example, wrapping the distal portion 62 of catheter 10 around a 1 mm diameter pin and comparing the outside diameter of the distal portion 62 while the distal portion 62 is wrapped about the pin, to the outside diameter of the distal portion 62 when the distal portion 62 is unwrapped, and unstressed. Thus, a kink resistance of 98% based on a first kink resistance test refers to decrease of only 2% in the outside diameter when the distal portion 62 is kinked.

In some embodiments, the catheter 10 was subjected not only to the first kink resistance test described above, but also to a BS EN 13868:2002 Kink Resistance Test commonly used to test kink resistance. In this test, two plates were spaced down to 3 mm apart, and the catheter 10 was wrapped about the two plates in a U-shaped formation. Flow rates were measured both prior to the catheter 10 being wrapped (when the catheter was a straight tube), as well as during the wrapping. The percentage decrease in flow rate between the measurements was calculated. It was determined that at least in some embodiments, the catheter 10 can have a percentage flow rate reduction of less than 50%. In some embodiments, the catheter 10 can have a percentage flow rate reduction of less than 40%. In some embodiments, the catheter 10 can have a percentage flow rate reduction of approximately 35%-38%.

This high level of kink resistance is advantageous, since other catheters often have much lower kink resistance, and thus encounter problems with keeping the inner lumen 22 wide enough to deliver intra luminal devices in the narrow, winding passageways of the human anatomy. The kink resistance of the distal portion 62 and the pushability of the catheter 10 overall (e.g., due to relatively stiff and easily maneuverable proximal, intermediate, and/or taper portions), make the catheter 10 an advantageous tool for use in delivering fluids and/or intraluminal devices in the tortuous pathways of the human body.

Furthermore, it has been found that the delivery force required to push intraluminal devices out of the catheter 10 can be advantageously low compared to other catheters. For example, during testing an embodiment of the intraluminal occluding device and delivery wire described in U.S. Patent Publication No. 2006/0271149, U.S. Patent Publication No. 2006/0271153, and U.S. Patent Publication No. 2009/0318947, was pushed through the distal portion 62 of an embodiment of the catheter 10 described above. The intraluminal delivery wire was pushed at 2 inches per minute through the most distal six inches of the catheter 10, and out the tip 64, moving in one inch strokes. For each one inch stroke of movement, the force (e.g., delivery force) required to push the delivery wire each one inch increment remained at equal to or less than 0.34 lbf. This low level of required delivery force is advantageous, since high levels of delivery force can suggest problems with friction, blocking, and/or difficulty in general in delivering an intra luminal device. Other values and ranges for delivery force of the catheter 10 are also possible.

Furthermore, the reinforced, multi-layered catheter 10 described above can advantageously withstand significant amounts of static and dynamic pressure. Static pressure, as described herein, corresponds to the burst strength of the catheter 10 while the lumen 28 is occluded at or near the distal tip 72. For example, in some embodiments, the catheter 10 can withstand at least 400 psi of static pressure, though other values and ranges are also possible. Dynamic pressure, as described herein, corresponds to the burst strength of the catheter 10 while the lumen 22 is not occluded. For example, in some embodiments, the catheter 10 can withstand at least 900 psi of dynamic pressure, though other values and ranges are also possible. It has been found that the catheter 10, if it did burst under static or dynamic pressure, would likely burst in the distal portion 62.

Furthermore, the multi-layered catheter 10 described above can exhibit an advantageous ratio of relative movement between the tip 72 and the proximal portion 12. For example, in some embodiments, the catheter 10 can exhibit a generally 1:1 movement response, meaning that if a surgeon or other medical personnel moves the proximal portion 12 of the catheter 10 one inch longitudinally along a central axis of a vessel inside the human body, the tip 72 will also generally move one inch longitudinally along a central axis of the vessel. In other embodiments this ratio can be different. For example, in some embodiments the ratio can be 1:2, or 2:1, or some other ratio. However, a 1:1 ratio can be desired, since the catheter 10 can often be used for applications in which it is desirable to move the catheter tip 72 at the same rate as the rest of the catheter. Furthermore, in some embodiments, and as described further below, the tip 72 can first be shaped or set by use of a mandrel, such that it has a bent profile as it moves along the axis of the vessel.

The 1:1 response described above can be repeatable and reliable, such that the surgeon or other medical personnel can confidently move the catheter 10 in and out of the vasculature of the human body knowing where the tip 72 is at all times. In catheters with more flexible intermediate and distal sections, it is often possible to have a tip or distal section that curls, bends, or twists unexpectedly, such that the correlation between movement of the proximal section and movement of the tip can vary greatly, making it difficult to assess the exact location of the tip, and to control movement of the tip.

Preparation and Use with Intraluminal Devices

The catheter 10 can be packaged by itself, or with other catheters. For example, a package or kit can contain a single catheter 10, for single use (e.g., disposable), or may include the catheter 10, a guidewire, and a delivery catheter that carries a stent or suitable occluding device as described elsewhere herein. The catheter 10 can be packaged in a packaging hoop.

Prior to using the catheter 10, and prior to removing the catheter 10 from the packaging hoop, the packaging hoop can be flushed with heparinized saline through a luer fitting connected to the end of the packaging hoop. If friction is felt when attempting to remove the catheter 10 from the packing hoop, one can conduct further flushing. The lumen 22 of the catheter can also be flushed with heparinized saline.

After flushing, the catheter 10 can be removed from the packaging hoop and inspected to make sure that it is undamaged. A shaping mandrel can be used to shape the tip 72 if desired. For example, a shaping mandrel can be inserted into the distal tip 72 of the catheter. The shaping mandrel can be bent to a desired shape. The mandrel and catheter tip 72 can be held directly over a steam source for approximately 30 seconds to set a shape for the tip. Other time lengths are also possible. The mandrel and catheter tip 72 can then be removed from the heat source to allow the mandrel and tip to cool in air or liquid prior to removing the mandrel. Once the catheter tip 72 and mandrel are cool, the mandrel can be removed and a guidewire can be inserted into the hub 26 and advanced through the lumen 22.

An appropriate guiding catheter can then be inserted into the human body, and a rotating hemostasis valve can be attached to the guiding catheter's luer connector, maintaining a continuous flush. Once the guiding catheter is in place, the catheter 10 and guide wire assembly can be introduced into the guiding catheter through a hemostasis sidearm adaptor, and the valve can be tightened around the catheter 10 to prevent backflow, but still allow movement of the catheter 10 through the valve. Although delivery through a guiding catheter is described herein, it will be appreciated that the catheter 10 may also be delivered without the use of a guiding catheter (or a guidewire, described further below).

The guidewire and catheter 10 can then be advanced through the guiding catheter to a selected target site in the human anatomy by alternately advancing the guidewire and then tracking the catheter 10 over the guidewire. Once the target location has been found (e.g., by referencing the marker band 70), the guidewire can be removed from the catheter 10. Fluid, an intraluminal device assembly, or some other material can then be inserted through the lumen 28 of the catheter 10. For example, an occluding device and delivery system such as that described in U.S. Patent Publication No. 2006/0271149, U.S. Patent Publication No. 2006/0271153, and U.S. Patent Publication No. 2009/0318947, the entirety of each of which is hereby incorporated by reference, can be inserted through the lumen 22 of catheter 10 and delivered to the tip 72. Similarly, a clot retrieval device and delivery system such as that described in U.S. Pat. No. 6,679,893 and U.S. Publication No. 2008/0269774, the entirety of each of which is hereby incorporated by reference, can be inserted through the lumen 22 of catheter 10 and delivered to the tip 72. Further details regarding devices, systems and methods that may be utilized with the catheter 10 are found in the aforementioned incorporated by reference applications.

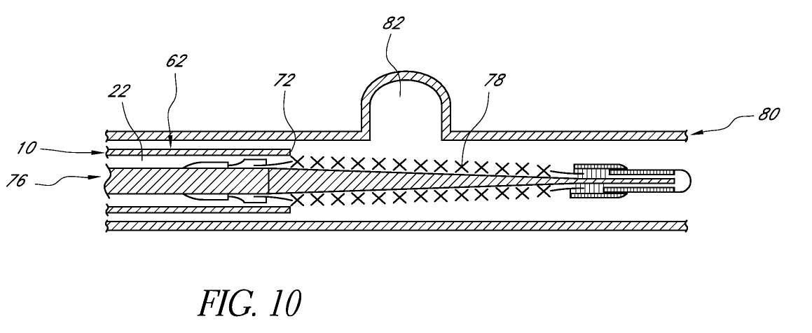

FIGS. 9 and 10 illustrate embodiments of the catheter 10 being used to deliver an occluding device delivery system 76. The occluding device delivery system 76 can include an expandable occluding device 78 such as a stent configured to be placed across an aneurysm that is delivered through the distal portion 62 of catheter 10, out the distal tip 72, and into the vasculature 80 adjacent a target location 82 (e.g. an aneurysm). In a preferred arrangement, the proximal portion 12 of catheter 10 can remain partially or entirely within the guiding catheter 84 during delivery, and the intermediate portion 36, taper portion 46, and distal portion 62 can extend distally of the guiding catheter 84. In some embodiments, a surgeon or other medical personnel can hold at least a portion of the proximal portion 12 outside of the body so as not to have his or her hand exposed during fluoroscopy. The occluding device 78 can be released at the target location 82, for example, and can be used to occlude blood flow into the aneurysm. The target location 82 (e.g. aneurysm) can be located at various locations in the human body. For example, in some embodiments an aneurysm can be located within at least one branch of the middle cerebral artery as shown in FIG. 9. The catheter 10 can be used to reach target locations (e.g. aneurysms) located elsewhere in the body as well, include but not limited to other arteries, branches, and blood vessels, such as arteries associated with the liver, and with the back of the head.

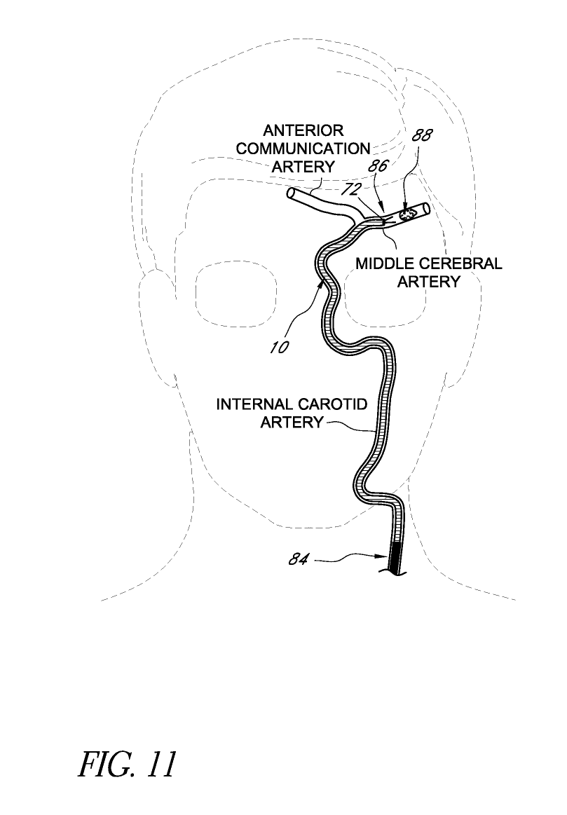

FIG. 11 illustrates an embodiment of the catheter 10 being used to delivery a clot retrieval device to remove a clot in the neurovasculature of the human brain. The catheter 10 can be delivered through a guiding catheter 84 into the internal carotid artery. The catheter 10 can be advanced until its distal tip 72 is located within the middle cerebral artery. In a preferred arrangement, the proximal portion 12 of catheter 10 can remain partially or entirely within the guiding catheter 84 during delivery, and the intermediate portion 36, taper portion 46, and distal portion 62 can extend distally of the guiding catheter 84.

As illustrated in FIG. 11, a clot retrieval device 86 can be delivered through the catheter 10, and advanced out the distal tip 72 proximal of a clot 88 in the middle cerebral artery. The clot retrieval device 86 can grab hold of the clot 88 and pull the clot 88 back towards the catheter 10 (e.g. pull the clot partially back into the catheter 10).

In some embodiments, prior to delivering the catheter 10 into the body, an introducer sheath (not shown) can be inserted into a patient's groin area. The guiding catheter 84 shown in FIGS. 9 and 11 can be inserted through the introducer sheath, and through the common carotid artery, such that the distal end of the guiding catheter extends into the internal carotid artery, and is located generally at the base of the skull. The guiding catheter 84 can be delivered, for example, through arteries and passageways too large for use of a microcatheter alone, since a microcatheter alone might bend, flop, or become entangled in a larger passageway. In some embodiments, the distal end of the guiding catheter 84 can extend to a location generally between the common carotid artery and the internal carotid artery, proximal to the carotid siphon. The catheter 10 can be inserted through the guiding catheter 84, and portions of the catheter 10 can extend out of the guiding catheter 84 as shown in FIGS. 9 and 11. In some embodiments, the size and length of the various catheter portions described above can facilitate insertion of the catheter 10 through the guiding catheter 84, and further through the narrow passageways of the internal carotid artery and middle cerebral artery. For example, the size and flexibility of the distal portion 62 (e.g. the outer diameter of the distal portion 62) can facilitate delivery of the catheter 10 through the middle cerebral artery, or other small arteries in the body, such that the catheter 10 can reach target locations deep within the vasculature. The size and length of the various catheter portions can additionally facilitate delivery of intraluminal devices and systems including but not limited to the clot retrieval device 86 and occluding device 78, to clots, aneurysms, or other target locations in the vasculature.

Once a procedure if finished (e.g. once a clot 88 is grabbed and pulled back at least partially into the catheter 10), the catheter 10 and intraluminal device or system can be removed from the neurovaculature. For example, in a preferred arrangement, the catheter 10 and intraluminal device or system can be pulled out together through the guiding catheter 84 together. Once they are removed from the body, the guiding catheter 84 can then be removed from the body. Other types of uses and methods of use for catheter 10 other than those described above are also possible.

Although these inventions have been disclosed in the context of certain preferred embodiments and examples, it will be understood by those skilled in the art that the present inventions extend beyond the specifically disclosed embodiments to other alternative embodiments and/or uses of the inventions and obvious modifications and equivalents thereof.

In addition, while several variations of the inventions have been shown and described in detail, other modifications, which are within the scope of these inventions, will be readily apparent to those of skill in the art based upon this disclosure. It is also contemplated that various combinations or sub-combinations of the specific features and aspects of the embodiments can be made and still fall within the scope of the inventions. It should be understood that various features and aspects of the disclosed embodiments can be combined with or substituted for one another in order to form varying modes of the disclosed inventions. Thus, it is intended that the scope of at least some of the present inventions herein disclosed should not be limited by the particular disclosed embodiments described above.

What is claimed is:

1. A variable flexibility catheter comprising:
an elongate tubular body having a proximal end, a distal end, and an inner lumen extending therethrough, the elongate tubular body comprising:
a proximal portion comprising:
a proximal portion outer jacket layer having a first stiffness, a braided stainless steel layer extending within the proximal portion outer jacket layer, a stainless steel coil layer extending within the braided material layer, and a low friction polymer PTFE layer extending within the stainless steel coil layer;
an intermediate portion distal to the proximal portion comprising:
an intermediate portion outer jacket layer having a second stiffness, a portion of the braided stainless steel layer extending within the intermediate portion outer jacket layer, a portion of the stainless steel coil layer extending within the braided stainless steel layer, and a portion of the low friction polymer PTFE layer extending within the stainless steel coil layer;
a taper portion distal to the intermediate portion comprising:
a tapered outer jacket layer having a third stiffness, a portion of the stainless steel coil layer extending within the tapered outer jacket layer, and a taper portion low friction polymer PTFE layer extending within the stainless steel coil layer;
a distal portion distal to the taper portion comprising:
a distal portion outer jacket layer having a fourth stiffness, a portion of the stainless steel coil layer extending within the distal portion outer jacket layer, and a portion of the low friction polymer PTFE layer extending within the stainless steel coil layer;
wherein the second stiffness is less than the first stiffness, the third stiffness is less than the second stiffness, and the fourth stiffness is less than the third stiffness; and
wherein the stainless steel coil layer extending within the distal portion has a coil pitch of approximately 0.007″ or more along at least one portion of the coil layer.

2. A variable flexibility catheter comprising:
an elongate tubular body having a proximal end, a distal end, and an inner lumen extending therethrough, the elongate tubular body comprising:
a proximal portion comprising:
a proximal portion outer jacket layer having a first stiffness, a braided material layer extending within the proximal portion outer jacket layer, a coil layer extending within the braided material layer, and a low friction polymer material layer extending within the coil layer;
an intermediate portion distal to the proximal portion comprising:
an intermediate portion outer jacket layer having a second stiffness, a portion of the braided material layer extending within the intermediate portion outer jacket layer, a portion of the coil layer extending within the braided material layer, and a portion of the low friction polymer material layer extending within the coil layer;
a taper portion distal to the intermediate portion comprising:
a tapered outer jacket layer having a third stiffness, a portion of the coil layer extending within the tapered outer jacket layer, and a taper portion low friction polymer material layer extending within the coil layer;
a distal portion distal to the taper portion comprising:
a distal portion outer jacket layer having a fourth stiffness, a portion of the coil layer extending within the distal portion outer jacket layer, and a portion of the low friction polymer material layer extending within the coil layer;
wherein the second stiffness is less than the first stiffness, the third stiffness is less than the second stiffness, and the fourth stiffness is less than the third stiffness.

3. The variable flexibility catheter of claim 2, wherein the taper portion further comprises a portion of the braided material layer extending within the taper portion outer jacket layer.

4. The variable flexibility catheter of claim 3, wherein the taper portion comprises a first segment having a constant first outside diameter and a second segment located distal of the first segment, the second segment having a second outside diameter that tapers distally along the second segment and is smaller than that of the first outside diameter.

5. The variable flexibility catheter of claim 4, wherein the braided material layer within the taper portion extends only along the first segment.

6. The variable flexibility catheter of claim 2, wherein the braided material layer has a uniform braid density along the entire length of the braided layer.

7. The variable flexibility catheter of claim 2, wherein the coil layer has a generally circular cross-section.

8. The variable flexibility catheter of claim 2, wherein the coil layer has a larger pitch in the distal portion than in the proximal portion.

9. The variable flexibility catheter of claim 2 further comprising a four-winged hub releasably attached to the proximal portion.

10. The variable flexibility catheter of claim 2, wherein the inner lumen has a generally constant diameter in the proximal, intermediate, taper, and distal portions.

11. A variable flexibility catheter comprising:
an elongate tubular body having a proximal end, a distal end, and an inner lumen extending therethrough, the elongate tubular body comprising:
a proximal portion comprising four layers;
a distal portion comprising three layers;
wherein the outer diameter of the distal portion is smaller than an outer diameter of the proximal portion, and wherein the distal portion is more flexible than the proximal portion.

12. The variable flexibility catheter of claim 11, wherein the proximal portion comprises a friction-resistant material layer, a metallic coil layer, a metallic braid layer, and a proximal portion outer jacket layer, the metallic coil layer located between the friction-resistant material layer and metallic braid layer, and the metallic braid layer located between the metallic coil layer and proximal portion outer jacket layer.

13. The variable flexibility catheter of claim 12, wherein the distal portion comprises a portion of the friction-resistant material layer and metallic coil layer, and further comprises a distal portion outer jacket layer.

14. The variable flexibility catheter of claim 13, wherein the distal portion outer jacket layer has a first stiffness, and the proximal portion outer jacket layer has a second stiffness greater than the first stiffness.

15. The variable flexibility catheter of claim 14, wherein the proximal portion has a stiffness of approximately 1.06 gm, and the distal portion has a stiffness of approximately 0.089 gm.

16. The variable flexibility catheter of claim 11, wherein the distal portion comprises a variable pitch metallic coil having a distal end, the metallic coil having a pitch between approximately 0.007″ and 0.012″ at the distal end.

17. The variable flexibility catheter of claim 11, wherein the distal portion comprises a kink resistance of at least 98%.

18. The variable flexibility catheter of claim 11, wherein catheter further comprises a four-winged hub releasably attached to the proximal portion.

19. The variable flexibility catheter of claim 11, wherein the distal portion comprises a distal tip, and wherein the catheter exhibits a generally a consistent 1:1 response ratio for movement of the proximal portion compared to movement of the tip.
